(19) United States
(12) Patent Application Publication
(10) Pub. No.: US 2011/0151310 A1
(43) Pub. Date: Jun. 23, 2011

(54) HIGH ENERGY DENSITY BATTERY FOR USE IN IMPLANTABLE MEDICAL DEVICES AND METHODS OF MANUFACTURE

(75) Inventor: Michael F. Pyszczek, Leroy, NY (US)

(73) Assignee: Balan Biomedical, Inc., West Henrietta, NY (US)

(21) Appl. No.: 13/058,642

(22) PCT Filed: Aug. 7, 2009
(86) PCT No.: PCT/US2009/053209

§ 371 Date: Feb. 11, 2011

Related U.S. Application Data
(60) Provisional application No. 61/088,762, filed on Aug. 14, 2008.

(51) Int. Cl. H01M 6/42; H01M 2/30; H01M 6/00; A61B 5/00; A61B 1/00

(52) U.S. Cl. 429/153; 429/181; 29/623.1; 29/592.1

(57) ABSTRACT

A high energy density battery is provided that improves energy density through efficient placement of the inter-plate connections within the battery enclosure. The placement of the current carrying leads in the high energy density battery allows for a greater volume of active material to be placed within the battery enclosure. This placement design can also be used to reduce the size of existing power sources. Methods for constructing high energy density batteries and methods for increasing the volumetric energy density of an implantable battery are also provided. The resulting high energy density battery can be used to power electronics associated with a variety of devices such as medical devices.

### CROSS-REFERENCE TO RELATED APPLICATIONS

This application claims priority to and the benefit of co-pending U.S. provisional patent application Ser. No. 61/088,762 entitled “High Energy Density Battery for Use in Implantable Medical Devices and Methods of Manufacture” filed Aug. 14, 2008, which is incorporated herein by reference in its entirety.

### TECHNICAL FIELD

The present invention relates to power sources, in particular, batteries that can be used in implantable medical devices. The invention also relates to methods for assembling batteries directly into an apparatus, in particular, into implantable medical devices. The invention further relates to implantable medical devices with integral power sources.

### BACKGROUND OF THE INVENTION

The batteries used in implantable medical devices have evolved over time to become more compact. Hat or prismatic shapes for batteries dominate this landscape because they can be designed to match the geometry of the implantable medical devices they power, such as cardiac pacemakers, implantable cardiofibrillators, and neurostimulation devices. Medical devices are typically designed with curved or rounded shapes approximating a circle, ellipse, or rectangle with beveled corners. The power source sealed within the device also conforms to the rounded shape to maximize energy density of the system. For example, in an implantable cardiac defibrillator with a circular shape, the power source would represent approximately one-half of the volume of the device and occupy a semi-circular area within the device.

The internal components of such batteries comprise flat electrodes that are arranged in stacks of individual, multiple plates (FIG. 1) (Keister, U.S. Pat. No. 4,830,940) that are electrically connected or continuous electrode strips that are folded together in a flat, jelly-roll or serpentine configuration (HG. 2) (Keister, U.S. Pat. No. 4,830,940), or a combination of both (FIG. 3) (Keister, U.S. Pat. No. 4,830,940). The designs of currently available batteries are dictated by the surface area of the electrodes on the batteries needed to meet the power requirements of the implantable medical device. Typical electrode surface areas for batteries designed for high current applications such as cardiofibrillators are in the range of 100 square centimeters.

The use of multiple plate designs can involve electrical connections from multiple anode plates and multiple cathode plates to separate current conductor (FIG. 4) (Keister, U.S. Pat. No. 4,830,940). These current conductors are then electrically connected to the battery case or one or more insulated electrical feed-throughs, providing positive and negative polarity terminals for the battery. In current methods of manufacture of batteries for implantable Medical devices, the battery enclosure or case can be a deep-drawn component with a lid assembly containing the insulated electrical feed-through (FIGS. 5A and 5B) (Youker, U.S. Pat. No. 7,344,800). Clamshell designs (FIG. 6), in which the enclosure is manufactured in two nearly symmetrical halves, are also used (Youker. U.S. Pat. No. 7,344,800).

In the construction of a semi-circular battery or one with substantially rounded corners to produce a physiologically-appropriate shape, a deep-drawn case as described by Haas (U.S. Pat. No. 6,040,082) is typically used. Other assembly methods utilize a clamshell design as disclosed by Paulot (U.S. Pat. No. 7,128,765), in which two halves are joined by a circumferential seam. A plate-like header described by Byland (U.S. Pat. No. 5,456,698) comprising one or more glass-to-metal seals is located on the flat side of the semicircular battery to complete the enclosure. The difficulties of utilizing the available volume within a battery of an irregular shape have, been disclosed by Probst (U.S. Pat. No. 6,946,220), which also discloses means for creating electrode components in shapes that conform to the battery ease. Interconnection of the components and the volume that the interconnection system requires, however, have not been addressed.

With both designs, i.e., the lidded assembly design and the clamshell design, electrical connections to the electrodes are typically made in the area of close proximity to the electrical feed-through. This is done to facilitate assembly of the battery. Prior art designs, however, cause a significant internal volume of the battery to be wasted. The energy density of the battery is a function of the volume of active material contained within. Reducing the size of the electrodes to accommodate the inter-plate electrical connections reduces energy density and thus reduces the service life of the power source. FIG. 6 depicts the volume of space typically required to accommodate the inter-plate connections (Youker, U.S. Pat. No. 7,344,800).

During assembly of a typical prior art battery, the internal components of the battery, usually comprising one or more planar anode and cathode components separated by non-conductive, separator material, are pre-assembled to make up an electrode stack assembly. The assembly is then inserted into the case, which can be lined internally with an insulative material. Once located within the ease, electrical connections to the glass-to-metal seals incorporated into the header assembly are made, typically through a welding operation. The header assembly is then pressed onto the case and welded in place to complete the enclosure. The battery is then activated through introduction of an electrolyte solution through a port in the header. The port is then sealed, again through a welding operation, to complete the hermetic sealing of the battery.

A drawback of this assembly method is that the area required to make the final electrical connections to the header assembly consumes a cross-section of the case with the highest volume, thus limiting the volume of active materials that can be contained in the enclosure.

There is therefore a need in the art for a battery use as a power source for implantable medical devices and other small-volume devices that improves energy density through more efficient placement of the inter-plate connections within the battery enclosure. There is also a need for greater volumes of active material to be contained within a sealed battery case to increase available energy.

Citation or identification of any reference in Section 2, or in any other section of this application, shall not be considered an admission that such reference is available as prior art to the present invention.

### SUMMARY OF THE INVENTION

A high energy density battery is provided that improves energy density through more efficient placement of the inter-plate connections within the battery enclosure. This improve dent is applicable to both primary (non-rechargeable) batteries as well as to secondary (rechargeable) batteries such as lithium ion batteries.

In one embodiment, the high energy density battery comprises:

a case comprising an outer surface, an inner surface and a case opening;

a header assembly inserted in the case opening, the header assembly comprising:
an electrical feed-through, the electrical feed-through comprising a terminal 3 in,
a glass-to-metal seal, and
a surrounding sidewall extending through the case opening to the outer surface and the inner surface of the case, the case opening being sized to receive the header assembly, with the header assembly surrounding, sidewall contacting, the case opening;

an electrode stack, the electrode stack comprising:
an anode layer, the anode layer comprising electrically conductive, chemically active material, an upper surface, and a lower surface,
an anode lead, the anode lead comprising electrically conductive material, an anode lead origin and an anode lead end, wherein:
   the anode lead origin is connected to the anode layer, and
   the anode lead end extends into the case and attaches to the terminal pin;

an insulative separator layer, the insulative separator layer comprising an upper surface and a lower surface;

a cathode layer, the cathode layer comprising electrically conductive, chemically active material, an upper surface and a lower surface;

a cathode lead, the cathode lead comprising electrically conductive material, a cathode lead origin and a cathode lead end, wherein:
the cathode lead origin is connected to the cathode layer, and
the cathode lead end extends into the case and attaches to the positive terminal;

a current collecting lead, the current collecting lead disposed between a cathode tab and the terminal pin, wherein the current collecting lead:
is electrically connected across the cathode layer of the electrode stack, and
is insulated with an insulative material; and

an electrolyte solution, the electrolyte solution disposed within the case and contacting the electrode stack.

In another embodiment, the battery case comprises a material selected from the group consisting of nickel, stainless steel, aluminum, titanium, glass and ceramic.

In another embodiment, the battery case is a deep-drawn battery case.

In another embodiment, the battery case is a multi-part or clam-shell case.

In another embodiment, the battery case is a liner or insulating bag.

In another embodiment, the high energy density battery comprises a plurality of electrode stacks, wherein the anode layer of one or more electrode stacks of the plurality is connected in series or in parallel to at least one other anode layer of an electrode stack of the plurality, and the cathode layer of one or more electrode stacks of the plurality is connected in series or in parallel to at least one other cathode layer of an electrode stack of the plurality.

In another embodiment, the electrode stacks are electrically connected to the current collecting lead.

in another embodiment, the header assembly comprises a plurality of electrical feed-throughs. In another embodiment, the lower surface of the insulative separator layer is disposed on the upper surface of the anode layer.

In another embodiment, the lower surface of the cathode layer is disposed on the upper surface of the insulative separator layer.

In another embodiment, the high density energy battery comprises a plurality of electrode stacks.

In another embodiment, the anode layer of one or more electrode stacks of the plurality is connected in series or in parallel to at least one other anode layer of an electrode stack of the plurality, and the cathode layer of one or more electrode stacks of the plurality is connected to at least one other cathode layer of an electrode stack of the plurality.

In another embodiment, the anode layer comprises a material selected from the group consisting, of a group IA metal or an alloy thereof (e.g., lithium, lithium compound), a group IIIA metal or an alloy thereof, and a carbonaceous material (e.g., carbon, graphite).

In another embodiment, the cathode layer comprises an active material selected from the group consisting of a fluorinated carbon material, a halogenated carbon material, a transition metal oxide, a transition metal sulfide, and a lithium insertion compound.

In another embodiment, the active material is an inter-dispersed pressed powder.

In another embodiment, the transition metal oxide is selected from the group consisting of Ag2O, Ag2O2, CuF2, Ag2CrO4, MnO2, V2O5, silver vanadium oxide, copper vanadium oxide, copper oxide, and copper silver vanadium oxide.

In another embodiment, the transition metal sulfide is selected, from the group consisting of TiS2, Cu2S, FeS, and FeS2.

In another embodiment the cathode layer comprises a lithium insertion compound.

In another embodiment, the electrode stack is positioned in the case to minimize unused volume within the case.

In another embodiment, the electrode stack has a flat, jelly-roll or serpentine configuration.

In another embodiment, the battery delivers at least about 20 joules in about 20 seconds or less.

In another embodiment, the battery delivers at least about 20 joules at least twice in a period of about 30 seconds.

A method for manufacturing a high energy density battery is also provided. In one embodiment, the method comprises:

a. providing a case,

b. providing an electrode stack assembly, wherein the electrode stack assembly comprises an anode layer, a cathode layer and a layer of separator material,

c. connecting the anode layer to the case with an anode connecting lead,

d. connecting the cathode layer to the insulated terminal pin with a cathode connecting lead,

e. positioning the anode connecting lead and the cathode connecting lead proximate to the center line of the stack and on the radiused or curved side of the stack;

f. electrically connecting the cathode layer to the positive current collecting lead,

g. electrically connecting the anode layers to a current collecting lead, wherein the current collecting lead is of sufficient length to extend from the side of the electrode stack where the connections are made to the opposing side of the stack assembly;

h. electrically connecting the cathode layers to a current collecting lead, wherein the current collecting lead is of sufficient length to extend from the side of the electrode stack where the connections are made to the opposing side of the stack assembly;

i. electrically connecting the positive current collecting lead to the feed-through pin;

j. electrically insulating the teed-through pin with a glass-to-metal seal, thereby providing the feed-through pin with positive polarity,

k. electrically connecting the anode layer to the negative current collecting, lead;

l. electrically connecting the current collecting lead to the case, thereby providing the case with negative polarity;

m. electrically connecting the positive current collecting lead to the glass-to-metal seal of the header assembly and to the electrode stack; and

n. attaching the feed-through pin to the case.

In another embodiment, the case can comprise an electrolyte fill port and the method can additionally comprise introducing an electrolyte solution into the electrolyte fill port of the case and hermetically sealing the electrolyte fill port.

A method for manufacturing a high energy density battery is also provided. In one embodiment, the method comprises:

providing a case, the case comprising an open portion;

attaching a header assembly to the case, the header assembly comprising;
an electrical feed-through, the electrical feed-through comprising a terminal pin, and
a glass-to-metal seal;

inserting an electrode stack into the case through the open portion, the electrode stack comprising:
an anode layer, the anode layer comprising electrically conductive, chemically active material, an upper surface, and a lower surface,
an anode lead, the anode lead comprising, electrically conductive material, an anode lead origin and an anode lead end, wherein:
   the anode lead origin is connected to the anode layer, and
   the anode lead end extends into the case and attaches to the terminal pin;
an insulative separator layer, the insulative separator layer comprising an upper surface and a lower surface;
a cathode layer, the cathode layer comprising electrically conductive, chemically active material, an upper surface and a lower surface;
a cathode lead, the cathode lead comprising electrically conductive material, a cathode lead origin and a cathode lead end, wherein:
   the cathode lead origin, is connected to the cathode layer,
   the cathode lead end extends from the case to the terminal pin,
   the cathode lead attaches to the terminal pin,

connecting a current collecting lead to the cathode lead and the terminal pin, wherein the current collecting lead:
is insulated with an insulative material and so that the current collecting lead extends across layers of the electrode stack;

placing an electrolyte solution inside the case;

placing a cover over the open portion of the case; and

hermetically sealing the cover to the case.

In one embodiment, the cathode layer is rolled or pressed.

In another embodiment, the cathode layer comprises a material formed by pressing or compressing a powdered active material.

An apparatus comprising a high energy density battery is provided. The apparatus can comprise an electrically powered device and a high energy density battery operatively connected to the electrically powered device.

A method for constructing an apparatus comprising the high energy density battery is also provided. In one embodiment, the method can comprise providing an electrically powered device, providing a high energy density battery, and operatively connecting the high energy density battery to the electrically powered device.

An apparatus comprising an electrically powered implantable medical device and the high energy density battery operatively connected to the electrically powered implantable medical device is provided. In one embodiment, the electrically powered implantable medical device is selected from the group consisting of cardiac rhythm management device, neurostimulation device, pump for dispensing drug or pharmaceutical composition, diagnostic sensor, regeneration and repair device, tissue repair device, and human interface device.

An apparatus comprising an electricity-generating device and the high energy density battery operatively connected to the electricity-generating device is also provided. In one embodiment, the electricity-generating device is selected from the group consisting of a photovoltaic array, a DC power supply, and a charging battery.

A method for constructing such an apparatus is also provided. In one embodiment, the method can comprise providing an electricity-generating device, providing a high energy density battery, and operatively connecting the high energy density battery to the electricity-generating device.

### BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated or enlarged to facilitate an understanding of the invention.

FIG. 1 is a diagram of a typical prior art plate electrode 10 comprising a current collecting grid 01 with an integral tab 20 for transmission of the current (,Keister, U.S. Pat. No. 4,830,940). Electrode active material 05 is adhered to the grid 01. The plate electrode 10 can be used as a single plate or one or several.

FIG. 2 is a diagram of a prior art serpentine electrode (Keister, U.S. Pat. No. 4,830,940). The serpentine electrode 30 comprises a current collecting grid 01 for current collection and mechanical support and an electrode active material 05. The multiple current conducting tabs 20 are formed as a part of or are connected to the current collecting grid 01.

FIG. 3 is a diagram of a prior art cell stack 02 comprising both electrically connected single plate electrodes 10 and a serpentine electrode 30 with current collecting tabs 20 enclosed in a deep drawn case 40 (Keister, U.S. Pat. No. 4,830,940).

FIG. 4 is a diagram of typical electrical connections of a prior art battery and the case volume required to accommodate them (Keister, U.S. Pat. No. 4,810,940). The plate electrodes 10 are electrically connected through individual tabs 20 to a common current collecting bus 70. The enclosure comprises the battery case 40 with the lid 50. The insulating glass seal 90 and terminal pin 60 provide electrical connection to an external circuit.

FIGS. 5A and 513 are diagrams of a prior art deep-drawn case and header in unassembled and assembled configurations (Youker, U.S. Pat. No. 7,344,800). The case 40 is matched to a lid 50 which comprises terminal pin 60 insulated from the lid by a glass seal 90. An opening 80 for introduction of liquid electrolyte is provided.

FIG. 6 is a diagram of a prior art clam-shell style battery case (Youker, U.S. Pat. No. 7,344,800). The cell stack 10 is located in one side of the battery case 40. The case is fitted with a terminal pin 60 electrically insulated from the case by a glass seal 90.

FIG. 7 is a diagram of a prior art high energy density battery with components (e.g., a generic layout of a pacemaker) and shows their positioning within a typical implantable medical device 100 comprising a battery 500, and electronic circuit 300, a inductive coil for communication 200, and a connector block 400 for attachment of output leads. Sec Section 5.1 for details.

FIG. 8 is a diagram of a prior art design for placement of electrode stack connections within a medical implantable battery (Keister. U.S. Pat. No. 4,830,940). Battery case 10. Electrode stack assembly 20. Cathode or positive plate(s) 21. Positive current collecting lead 22. Anode or negative plate(s) 23. Negative current collecting, lead 24. Header assembly 30. Glass-to-metal seal 40. Feed-through pin 50. Enclosure 60. Fill port 70. Metal ball 80. See Section 5.1 for details.

FIG. 9 is a diagram of one embodiment of the invention, in which the electrode (i.e., anode and cathode) lead placement enables greater utilization of the internal volume of the battery enclosure. Battery case 10. Electrode stack assembly 20. Cathode or positive plate(s) 21. Positive current collecting lead 22. Anode or negative plate(s) 23. Negative current collecting lead 24. Header assembly 30. Glass-to-metal seal 40. Feed-through pin 50. Enclosure 60. Fill port 70. Metal ball 80. See Section 5.1 for details.

FIG. 10 is a diagram of the prior art method for welding a current collecting lead 40 to au electrical feed-through pin 10 incorporated in an insulative glass-to-metal seal (Keister, U.S. Pat. No. 4,830,940). Electrically insulative material 20. Outer metallic ferrule 30. Portion of feed-through pin extending below the glass-to-metal seal 50. Weld zone 60. See Section 5.2.1 for details.

FIG. 11 is a diagram of an embodiment of the method provided herein for placement of a current collecting lead 40 to an electrical feed-through pin 10 incorporated in an insulative glass-to-metal seal which reduces the internal volume of the battery required to accommodate the connection. Electrically insulative material 20. Outer metallic ferrule 30. Portion of feed-through pin extending below the glass-to-metal seal 50. Weld zone 60. See Section 5.2.2 for details.

### DETAILED DESCRIPTION OF THE INVENTION

A high energy density battery is provided that that improves energy density through efficient placement of the inter-plate connections within the battery enclosure. The placement of the current carrying leads in the high energy density battery allows for a greater volume of active material to be placed within the battery enclosure. This placement design can also be used to reduce the size of existing power sources. Methods for constructing high energy density batteries and methods for increasing the volumetric energy density of a battery (e.g., an implantable battery) are also provided. The resulting high energy density battery can be used to power electronics associated with a variety of devices such as medical devices.

The high energy density battery provides a significant improvement over the prior art by enabling greater volumes of active material to be contained within the case of a new or existing battery design. In certain embodiments, the volume of the high energy density battery is reduced significantly while maintaining the same level of stored energy. The high energy density battery provided herein addresses the limitations of prior art batteries by offering, an increased volume of active material that can be contained within a sealed battery enclosure.

In one embodiment, the high energy density battery comprises:

a case comprising an outer surface, an inner surface and a case opening;

a header assembly inserted in the case opening, the header assembly comprising:
an electrical feed-through, the electrical feed-through comprising a terminal pin,
   a glass-to-metal seal, and
   a surrounding sidewall extending through the case opening to the outer surface and the inner surface of the case, the case opening being sized to receive the header assembly, with the header assembly surrounding sidewall contacting the case opening;

an electrode stack, the electrode stack comprising:
an anode layer, the anode layer comprising electrically conductive, chemically active material, an upper surface, and a lower surface.
an anode lead, the anode lead comprising electrically conductive material, an anode lead origin and an anode lead end, wherein:
   the anode lead origin is connected to the anode layer, and
   the anode lead end extends into the case and attaches to the terminal pin;

an insulative separator layer, the insulative separator layer comprising an upper surface and a lower surface;

a cathode layer, the cathode layer comprising electrically conductive, chemically active material, an upper surface and a lower surface;

a cathode lead, the cathode lead comprising, electrically conductive material, a cathode lead origin and a cathode lead end, wherein:
the cathode lead origin is connected to the cathode layer, and
the cathode lead end extends into the case and attaches to the positive terminal;

a current collecting lead, the current collecting, lead disposed between a cathode tab and the terminal pin, wherein the current collecting lead;
is electrically connected across the cathode layer of the electrode stack, and
is insulated with an insulative material; and

an electrolyte solution, the electrolyte solution disposed within the case and contacting the electrode stack.

In one embodiment, the high energy density battery is a primary (non-rechargeable) battery. In another embodiment, the high energy density battery is a secondary (rechargeable) battery.

In another embodiment, the battery case comprises a material selected from the group consisting of nickel, stainless steel, aluminum, and titanium or a glass or ceramic material.

In another embodiment, the battery case is a deep-drawn battery case.

In another embodiment, the battery case is a multi-part or clam-shell case.

In another embodiment, the battery case is a liner or insulating bag.

In another embodiment, the high energy density battery comprises a plurality of electrode stacks, wherein the anode layer of one or more electrode stacks of the plurality is connected in series or in parallel to at least one other anode layer of an electrode stack of the plurality, and the cathode layer of one or more electrode stacks of the plurality is connected in series or in parallel to at least one other cathode layer of an electrode stack of the plurality.

In another embodiment, the electrode stacks are electrically connected to the current collecting lead.

In another embodiment, the header assembly comprises a plurality of electrical feed-throughs. In another embodiment, the lower surface of the insulative separator layer is disposed on the upper surface of the anode layer.

In another embodiment, the lower surface of the cathode layer is disposed on the upper surface of the insulative separator layer.

In another embodiment, the high density energy battery comprises a plurality of electrode stacks.

In another embodiment, the anode layer of one or more electrode stacks of the plurality is connected in series or in parallel to at least one other anode layer of an electrode stack of the plurality, and the cathode layer of one or more electrode stacks of the plurality is connected to at least one other cathode layer of an electrode stack of the plurality.

In another embodiment, the anode layer comprises lithium or other group IA or IIIA metal or alloy thereof, a lithium compound, carbon, graphite, or another carbonaceous material.

In another embodiment, the cathode layer comprises an active material selected from the group consisting of a fluorinated carbon material, a halogenated carbon material, a transition metal oxide and a transition metal sulfide.

In another embodiment, the active material is an inter-dispersed pressed powder.

In another embodiment, the transition metal oxide is selected from the group consisting of Ag2O, Ag2O2, CuF2, Ag2CrO4, MnO2, V2O5, silver vanadium oxide, copper vanadium oxide, copper oxide, and copper silver vanadium oxide.

In another embodiment, the transition metal sulfide is selected, from the group consisting of TiS2, Cu2S, FeS, and FeS2.

In another embodiment the cathode layer comprises a lithium insertion compound.

In another embodiment, the electrode stack is positioned in the case to minimize unused volume within the case.

In another embodiment, the electrode stack has a flat, jelly-roll or serpentine configuration.

In another embodiment, the battery delivers at least about 20 joules in about 20 seconds or less.

in another embodiment, the battery delivers at least about 20 joules at least twice in a period of about 30 seconds.

A method for manufacturing a high energy density battery is also provided. In one embodiment, the method comprises:

a. providing a ease;

b. providing an electrode stack assembly, wherein the electrode stack assembly comprises an anode layer, a cathode layer and a layer of separator material;

c. connecting the anode layer to the case with an anode connecting lead,

d. connecting the cathode layer to the insulated terminal pin with a cathode connecting lead;

e. positioning the anode connecting lead and the cathode connecting lead proximate to the center line of the stack and on the radiused or curved side of the stack;

f. electrically connecting the cathode layer to the positive current collecting lead;

g. electrically connecting the anode layers to a current collecting lead, wherein the current collecting lead is of sufficient length to extend from the side of the electrode stack where the connections are made to the opposing side of the stack assembly;

h. electrically connecting, the cathode layers to a current collecting lead, wherein the current collecting lead is of sufficient length to extend from the side of the electrode stack where the connections are made to the opposing side of the stack assembly;

i. electrically connecting the positive current collecting lead to the trod-through pin;

j. electrically insulating the teed-through pin with a glass-to-metal seal, thereby providing the feed-through pin with positive polarity;

k. electrically connecting the anode layer to the negative current collecting, lead;

l. electrically connecting the current collecting lead to the case, thereby providing the case with negative polarity;

m. electrically connecting the positive current collecting lead to the glass-to-metal seal of the header assembly and to the electrode stack;

n. attaching the feed-through pin to the case.

In another embodiment, the method additionally comprises introducing an electrolyte solution into an electrolyte fill port of the case and hermetically sealing the electrolyte fill port.

A method for manufacturing a high energy density battery is also provided. In one embodiment, the method comprises:

providing a case, the case comprising an open portion;

attaching a header assembly to the case, the header assembly comprising:
an electrical feed-through, the electrical feed-through comprising a terminal pin, and
a glass-to-metal seal;

inserting an electrode stack into the case through the open portion, the electrode stack comprising:
an anode layer, the anode layer comprising electrically conductive, chemically active material, an upper surface, and a lower surface,
an anode lead, the anode lead comprising, electrically conductive material, an anode lead origin and an anode lead end, wherein:
   the anode lead origin is connected to the anode layer, and
   the anode lead end extends into the case and attaches to the terminal pin,
an insulative separator layer, the insulative separator layer comprising an upper surface and a lower surface,
a cathode layer, the cathode layer comprising electrically conductive, chemically active material, an upper surface and a lower surface,
a cathode lead, the cathode lead comprising electrically conductive material, a cathode lead origin and a cathode lead end, wherein:
   the cathode lead origin, is connected to the cathode layer,
   the cathode lead end extends from the case to the terminal pin, and
   the cathode lead attaches to the terminal pin,

connecting a current collecting, lead to the cathode lead and the terminal pin, wherein the current collecting lead:
is insulated with an insulative material and so that the current collecting lead:
extends across layers of the electrode stack;

placing an electrolyte solution inside the case;

placing a cover over the open portion of the case; and

hermetically sealing the cover to the case.

In one embodiment, the cathode layer is rolled or pressed.

In another embodiment, the cathode layer comprises a material formed by pressing or compressing a powdered active material.

An apparatus is also provided. In one embodiment, the apparatus comprises an electrically powered implantable medical device and the high energy density battery operatively connected to the electrically powered implantable medical device.

In another embodiment, the electrically powered device is selected from the group consisting of cardiac rhythm management device, neurostimulation device, pump for dispensing drug or pharmaceutical composition, diagnostic sensor, regeneration and repair device, tissue repair device, and human interface device.

Another apparatus is also provided. In one embodiment, the apparatus comprises an electricity-generating device and the high energy density battery operatively connected to the electricity-generating device.

In another embodiment, the electricity-generating device is selected from the group consisting of a photovoltaic array, a DC power supply, and a charging battery.

The high energy density battery provides a significant improvement over the prior art by enabling greater volumes of active material to be contained within the case of a new or existing, battery design. In certain embodiments, the volume of the high energy density battery is reduced significantly while maintaining the same level of stored energy. The high energy density battery provided herein addresses the limitations of prior art batteries by offering an increased volume of active material that can be contained within a scaled battery enclosure.

Also provided is an apparatus incorporating a high energy density battery and a method for constructing an apparatus incorporating the high energy density battery. The apparatus can comprise an electrically powered device and a high energy density battery operatively connected to the electrically powered device.

An apparatus comprising an electricity-generating device and a battery operatively connected to the electricity-generating, device, and a method for constructing such an apparatus are also provided. The electricity-generating device can be, for example, a photovoltaic array, a DC power supply, or a charging battery.

For clarity of disclosure, and not by way of limitation, the detailed description of the invention is divided into the subsections set forth below.

5.1 High Energy Density Battery

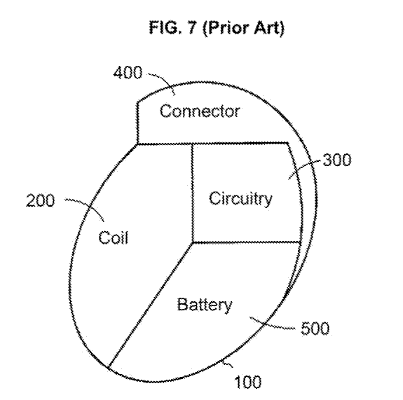

Implantable medical batteries are typically designed to fit the rounded or curved shape of the medical device to which they supply electrical energy. Prior art high energy density batteries are typically enclosed within the device case and can comprise a significant portion of the overall volume of the device. FIG. 7 shows a schematic of a prior art design for an implantable medical device 100 powered by a prior art high energy density battery. Within the device case are located the microelectronics 200 that generate the required electrical pulse, a coil 300 for telemetry or recharging, and the battery 500. External to the device case is an electrode connection block which is used to attach the current carrying leads to the device 400. This prior art design for a battery for an implantable medical device, as is typical of all prior art designs, occupies a significant portion of the device volume. Reducing the volume of the battery is desirable because it enables the implantable device to be smaller and more comfortable to the patient.

FIG. 9 shoves one embodiment of the high energy density battery provided by the invention. In contrast to the prior art battery, the volume of the high energy density battery of the invention is significantly reduced. In this embodiment, the high energy density battery has negative case polarity and the insulated teed-through pin has positive polarity.

In certain embodiments, the electrode stack assembly 20 can comprise one or more electrode plates (i.e., the anode and cathode plates) and layers of separator material. The electrode stack 20 can be made larger and can contain a greater volume of active material as compared with prior art batteries. Furthermore, the design of the electrode stack assembly and the positioning of the electrically conductive leads can allow for higher efficiency of the available internal battery volume.

The connections to the cathode plate 21 and anode plate 23 are positioned in the area of the electrode stack closer to the center line of the stack and on the radiused or curved side of the stack, which permits, in this embodiment, the electrode stack to be placed in the bottom of the battery case 10. The electrical connections for each of the one or more anode and cathode components can be placed so that they are located substantially closer to the center line of the component than is common in prior art batteries.

The cathode or positive plate(s) 21 is electrically connected to the positive current collecting lead 22. The lead 22 can be electrically insulated and can extend from the electrode plate connections 21 to the top of the cell case.

Electrical connection between the common polarity electrodes and a current collecting lead can be made with the lead being of sufficient length (as easily determined by one of skill in the art) to extend from the side of the electrode stack where the connections are made to the opposing side of the stack assembly. The lead may be coated or covered with an insulative material as appropriate and commonly known in the art.

The positive current collecting lead 22 is electrically attached to the feed-through pin 50. The teed-through pin can be electrically insulated by means of a glass-to-metal seal 40, thus providing the pin with a positive polarity.

The anode or negative plate(s) 23 is electrically connected to the negative current collecting lead 24. The negative lead 24 can extend from the anode plate connections 23 to the top of the electrode stack. The current collecting lead 24 is electrically attached to the case wall, thus providing the enclosure with a negative polarity. In one embodiment, the negative lead can extend out of the case and can be captured or pinched in the seam created when the header assembly is positioned.

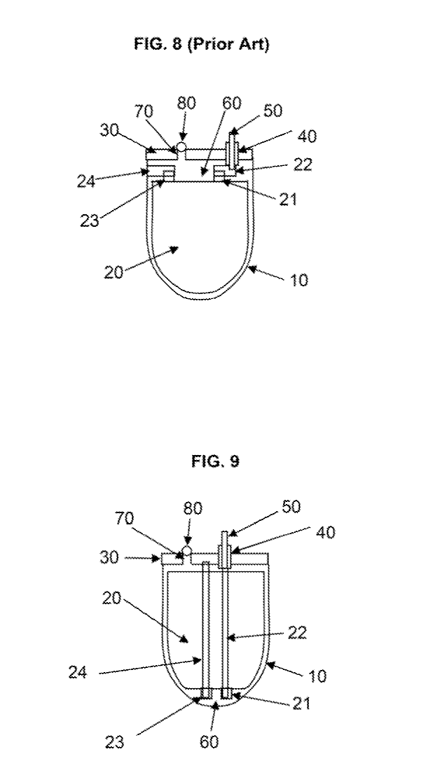

The header assembly 30 can comprise the glass-to-metal seal 40 which is attached to the electrode stack 20 by means of the positive current collecting lead 22. To accommodate the leads and connections, a segment of the internal volume of the enclosure 60 remains vacant. It is obvious to those skilled in the art that the volume of vacant space in the enclosure 60 required by the present invention is substantially smaller than that required by the prior art shown in FIG. 8 (Keister, U.S. Pat. No. 4,830,940).

The high energy density batter of the invention can comprise an electrolyte solution. Any electrolyte solution known in the art can be used. With reference to FIG. 9, an electrolyte solution can then be added through the fill port 70, which can be sealed, e.g. hermetically sealed, with a metal ball 80 or other sealing device known in the art. Omitted from FIG. 9 for purposes of clarity are the electrically insulative materials to make the battery operational, and known to those skilled in the art.

In another embodiment, the case or battery enclosure for the high energy density battery enclosure can comprise a deep-drawn metal can. In another embodiment, the header assembly can be sized to fit the open end of the case. The electrode stack can be placed in an insulating bag which is then inserted into the deep-drawn can with current collection tabs placed along the outer surface of the electrode stack and electrically attached to the header.

In still another embodiment of the high energy density battery, a clamshell, case comprising two halves of a battery enclosure can be used to contain an electrode stack with current-collection leads placed along the outer surface of the electrode stack. The stack can then be inserted into an electrical insulating hag which is placed in one half of the split case design. Electrical connections can be made to the feed-through pin of the glass to metal seal incorporated into the half of the clamshell case containing the electrode stack. The opposite polarity connection is made to the other, matching case half.

In another embodiment, the high energy density battery can be a primary lithium battery. The anode can comprise a lithium or other group IA or IIIA metal or alloy thereof, and the cathode can comprise a transition metal oxide or combination of transition metal oxides or sulfides including but not limited to Ag₂O, Ag₂O₂, CuF₂, Ag₂CrO₄, MnO₂, V₂O₅, TiS₂, Cu₂S, FeS, FeS₂, silver vanadium oxide, copper vanadium oxide, copper oxide, and copper silver vanadium oxide.

In another embodiment, the anode can comprise lithium or other group IA or IIIA metal or alloy thereof and the cathode can comprise a fluorinated carbon or a mixed halogenated carbon material.

In one embodiment, the high energy density battery is a primary non-rechargeable) battery.

In another embodiment, the high energy density battery is a secondary (rechargeable) battery. The anode can comprise, for example, a lithium compound, carbon, graphite, or another carbonaceous material. The cathode layer can comprise a lithium insertion compound.

The high energy density battery provided herein provides an improvement over the prior art by enabling a greater portion of the internal volume of an implantable medical battery case to be used to store active material used in the electrochemical process. This can be accomplished through the placement of the internal leads used to carry electrical current. The result of the more efficient use of the available volume within the battery case can be a reduction in the overall size of the battery and hence, a reduction in the size of the implantable medical device.

The high energy density battery can be used in a medical device that has an optimal physiological shape. The internal power source provided by the high energy density battery can use the optimal physiological shape as a basis for improving energy density. Methods for designing a medical device, e.g., an implantable medical device, with an optimal physiological shape are well known in the art.

5.2 Methods for Constructing High Energy Density Batteries

5.2.1 Prior Art Methods for Constructing High Energy Density Batteries

During assembly of a typical prior art battery, the internal components of the battery, usually comprising one or more planar anode and cathode components separated by non-conductive separator material, are pre-assembled to make up an electrode stack assembly. The assembly is then inserted into the case, which can be lined internally with an insulative material. Once located within the case, electrical connections to the glass-to-metal seals incorporated into the header assembly are made, typically through a welding operation. The header assembly is then pressed onto the case and welded in place to complete the enclosure. The battery is then activated through introduction of an electrolyte solution through a port in the header. The port is then sealed, again through a welding operation to complete the hermetic scaling of the battery.

A prior art battery for an implantable medical device is shown in FIG. 8 and is disclosed by Keister U.S. Pat. No. 4,830,940). This prior art battery employs a deep-drawn metal case 10 as one component of a battery enclosure. The polarity of the case is negative and the polarity of the insulated feed-through pin is positive. The electrode stack assembly 20 comprises one or more electrode plates and layers of separator material. The cathode or positive plate(s) 21 are electrically connected to the positive current collecting lead 22 by welding. The current collecting lead is electrically connected, typically through welding, to the feed-through pin 50, which is electrically insulated by a glass-to-metal seal 40, thus providing the pin with a positive polarity.

In this prior art battery, the anode or negative plate(s) 23 is electrically connected to the negative current collecting lead 24 by means of a welding operation. The current collecting lead is electrically attached, typically through a welding operation to the case wall, thus providing the enclosure with a negative polarity.

The electrode of the prior art design may incorporate an electrical current carrying lead formed as a unitary component along with the cathode material support grid. The unitary lead can be positioning according to methods well known in the art, by forming as part of the current collector or through addition of another component such as a conductive ribbon.

The header assembly 30 of the prior art battery comprises the glass-to-metal seal 40. The glass-to-metal seal is attached to the electrode stack 20 by means of the positive current collecting lead 22 and feed-through pin 50, is pressed into the case 10, and is welded. To accommodate the leads and connections, a segment of the internal volume of the enclosure 60 remains vacant. The battery is completed through addition of an electrolyte solution through the fill port 70 which is then scaled with a metal ball 80 placed over the hole and welded in place to render the enclosure hermetic. Omitted for purposes of clarity are the electrically insulative materials required to make the battery operational, and known to those skilled in the art.

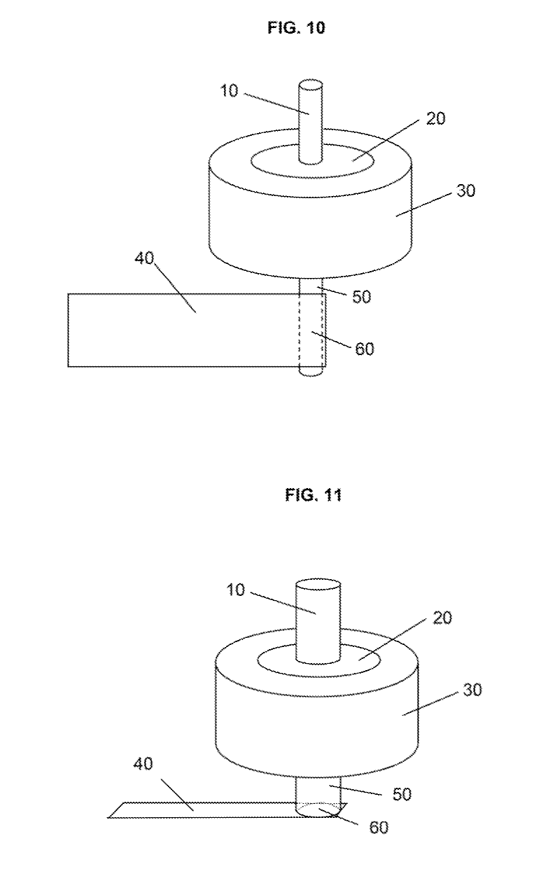

FIG. 10 illustrates a prior art method that is used to electrically join a current collecting lead 40 to a feed-through pin 10 (Greatbatch, U.S. Pat. No. 3,874,929). The electrical feed-through pin 10 is insulated from an outer metallic ferrule 30 by means of an electrically insulative material 20 such as a glass or ceramic. The current conducting lead 40 which consists of a metal ribbon is placed against the feed-through pin 10 on the portion of the pin extending below 50 the glass-to-metal seal, and welded by resistance welding the weld zone 60.

5.2.2 Methods for Constructing High Energy Density Batteries

Methods for constructing high energy density batteries are provided that enable a greater portion of the internal volume of an implantable medical battery case to be used to store active material used in the electrochemical process. One embodiment of the high energy density battery of the invention is illustrated in FIG. 9. In this embodiment, the high energy density battery has negative case polarity and the insulated feed-through pin has positive polarity.

A deep-drawn metal case 10 can be used as the case or battery enclosure. Any suitable metal known in the art, such as stainless steel, can be used to form the battery case. Materials known in the art such as titanium can also be used.

In certain embodiments, the electrode stack assembly 20 can comprise one or more electrode plates and layers of separator material. The electrode stack 20 can be made larger and can contain a greater volume of active material as compared with prior art batteries. Furthermore, the design of the electrode stack assembly and the positioning of the electrically conductive leads can allow for higher efficiency of the available internal battery volume.

The individual electrode plates, i.e., the anode and cathode plates, and thus the electrode stack, can be formed according to methods well known in the art, so that the electrode plate connections 21, 23 are positioned in the area of the electrode stack closer to the center line of the stack and on the radiused or curved side of the stack, which permits, in this embodiment, the electrode stack to be placed in the bottom of the battery case 10. The electrical connections thr each of the one or more anode and cathode components can be placed so that they are located substantially closer to the center line of the component than is common in prior art batteries.

The cathode or positive plate(s) 21 can be electrically connected to the positive current collecting lead 22 by any connection method known in the art, e.g., by a welding operation. The lead 22 can be electrically insulated and can extend from the electrode plate connections 21 to the top of the cell case.

Electrical connection between the common polarity electrodes and a current collecting lead can be made with the lead being of sufficient length to extend from the side of the electrode stack where the connections are made to the opposing side of the stack assembly. The lead may be coated or covered with an insulative material as appropriate and commonly known in the art.

The positive current collecting lead 22 can be electrically attached, far example, through methods well known in the art such as welding, to the feed-through pin 50. The feed-through pin can be electrically insulated by means of a glass-to-metal seal 40, thus providing the pin with a positive polarity.

The anode or negative plate(s) 23 can be electrically connected to the negative current collecting lead 24 by any connection method known in the art, e.g., by a welding operation. The negative lead 24 can extend from the anode plate connections 23 to the top of the electrode stack. The current collecting lead 24 can be electrically attached, by any connection method known in the art, e.g., by a welding operation, to the case wall, thus providing the enclosure with a negative polarity. In one embodiment, the negative lead can be extended out of the case and captured or pinched in the seam created when the header assembly is positioned.

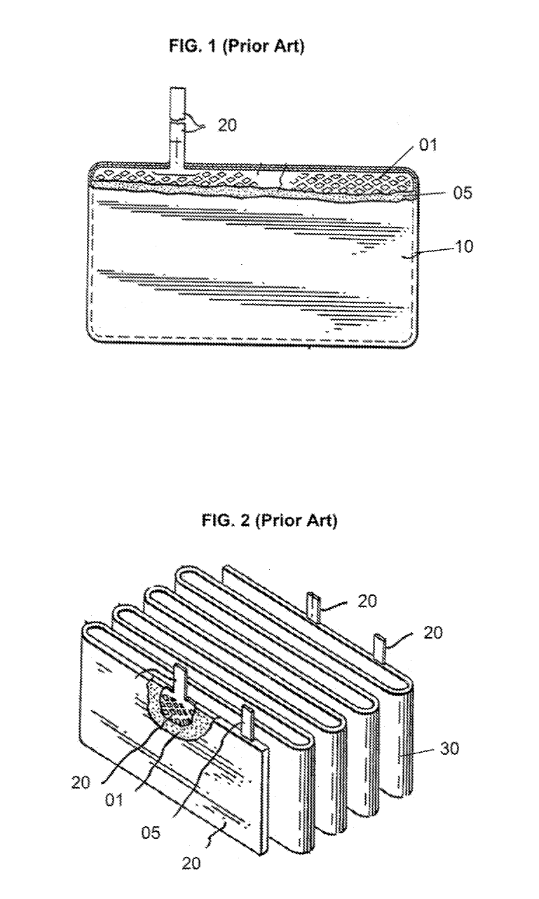

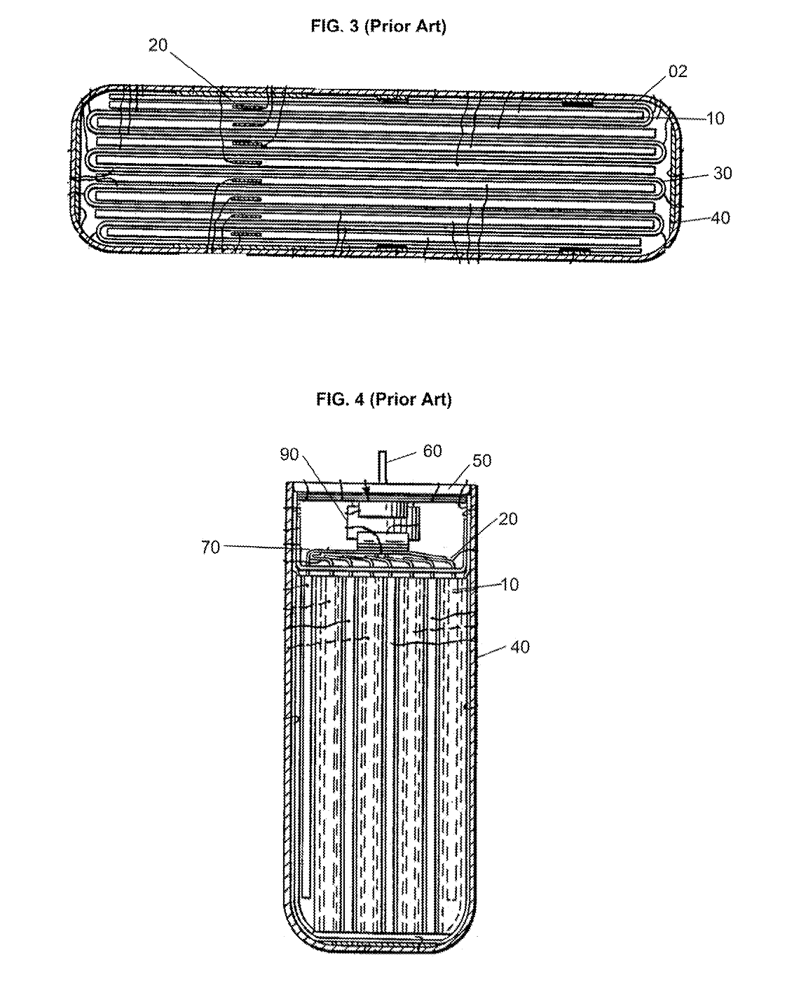

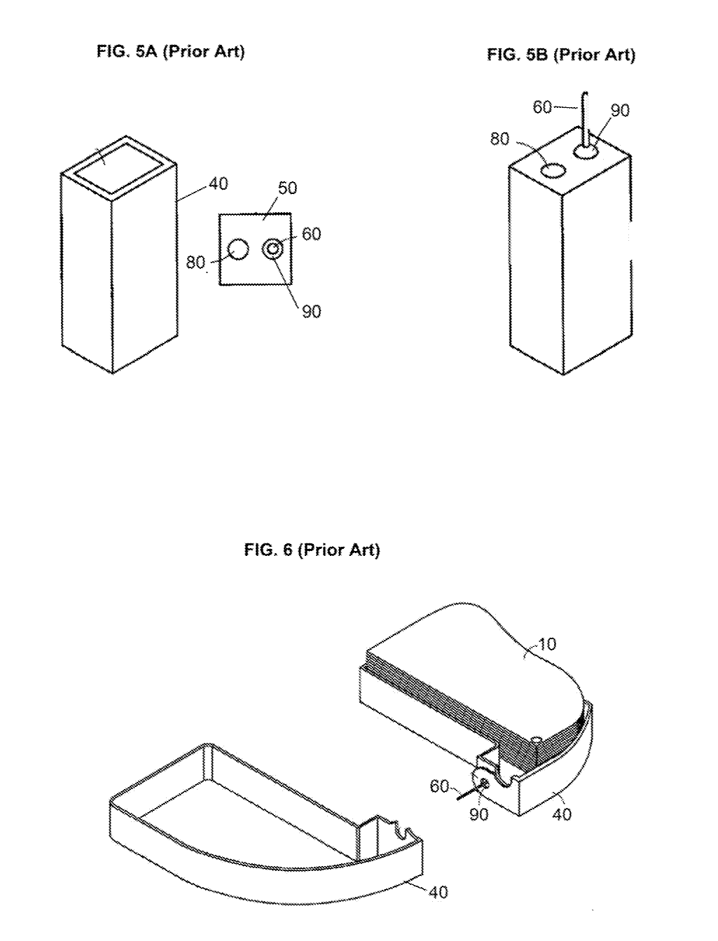

The header assembly 30 can comprise the glass-to-metal seal 40 which is now attached to the electrode stack 20 by means of the positive current collecting lead 22 and feed-through pin 50 is pressed into the case 10 and welded. To accommodate the leads and connections, a segment of the internal volume of the enclosure 60 remains vacant. It is obvious to those skilled in the art that the volume of vacant space 60 required by the present invention is substantially smaller than that required by the prior art shown in FIG. 2 (Keister, U.S. Pat. No. 4,830,940).

In certain embodiments, final assembly steps can comprise introducing an electrolyte, solution and hermetically sealing the electrolyte fill port. The final assembly steps can be according to currently accepted practices for medical battery manufacture known in the art. With reference to FIG. 9, an electrolyte solution can then be added through the fill port 70, which can be sealed, with a metal ball 80 or other sealing device known in the art, which is placed over the hole and welded, soldered, affixed in place, or sealed by other art-known methods, to render the enclosure hermetic.

The high energy density battery is insulated with electrically insulative materials, which are well known to those skilled in the art.

FIG. 11 illustrates an embodiment of the method of constructing a high energy density battery, which can comprise an improved method of joining a current collecting lead 40 to a feed-through pin 10 in a manner that confers distinct advantages to the high energy density battery such as reduced size. The feed-through pin 10 can be fabricated with a diameter suitable to provide a weld zone 60 on the circular cross-section of the pin. The electrical feed-through pin 10 can be insulated from an outer metallic ferrule 30 by means of an electrically insulative material 20 known in the art such as a glass or ceramic. The current conducting lead 40, which can consist, in certain embodiments, of a metal ribbon can be placed against the flat end of the portion of the feed-through pin extending below 50 the glass-to-metal seal, and can be welded by resistance welding, soldering, brazing, or other forms of metal joining in the weld zone 60.

It will be apparent to those skilled in the art that the section of the feed-through pin extending below the glass-to-metal seal 50 can be made shorter or longer than is shown in FIG. 11. This flexibility in the length of the feed-through pin can enable it to be optimally located to reduce cell volume.

In one embodiment, the electrode stack can be made larger since the connections are advantageously located in a smaller cross-sectional area of the battery case.

The electrode anode and cathode) leads can then be attached to the electrical feed-throughs located on the header assembly. Further optimization of internal volume is achieved through the use of an electrical feed-through pin of sufficient diameter to allow the lead to be attached according to methods well known in the art (e.g., welded, soldered, or mechanically joined), onto the flat surface on the end of the pin. In embodiments in which the polarity of the ease is either positive of negative, the anode or cathode lead can be extended out of the case and captured in the seam between the case and header assembly during placement of the header assembly. The header assembly can be seam-welded or mechanically joined, which completes the electrical connection from the lead to the case.

The method provided herein also has the advantage of falling within generally accepted practices known in the art for case manufacture, namely those that are deep-drawn enclosures or clamshell case designs. Further in accord with generally accepted practices for case manufacture known in the art, a plate-like header assembly can be used to accommodate the insulated electrical feed-throughs and electrolyte fill port.

Electrical connection to the feed-through pin of the glass-to-metal seal located in the header assembly can then be made. The opposite polarity electrode lead can be welded or attached by other means known in the art to the inside of the battery case.

In another embodiment, the case or battery enclosure for the high energy density battery enclosure can comprise a deep-drawn metal can. In another embodiment, the header assembly can be sized to fit the open end of the case.

In another embodiment, high energy density battery can comprise an insulating bag, made from any material known in the art, and the electrode stack can be placed in the insulating bag. The insulating bag can then be inserted into the battery enclosure (e.g., a deep-drawn can) with current collection tabs placed along the outer surface of the electrode stack and electrically attached to the header by welding one current collecting tab to the feed-through pin of the glass-to-metal seal located in the header assembly and the other opposite polarity lead to the header. The completed electrode stack can be inserted into the case with the connection end of the electrode stack leading into the case. The header assembly can be pressed onto the open end of the deep-drawn can and welded in place.

An electrolyte solution is introduced through the fill port, which can then sealed with a metal ball or plate placed over the hole and welded in place to render the enclosure hermetic.

In still an her embodiment of the high energy density battery, a clamshell case comprising two halves of a battery enclosure can be used to contain an electrode stack with current-collection leads placed along the outer surface of the electrode stack. The stack can then be inserted into an electrical insulating hag which is placed in one half of the split case design. Electrical connections can be made to the teed-through pin of the glass to metal seal incorporated into the half of the clamshell case containing the electrode stack. The Opposite polarity connection is made to the other, matching case half. The matching half of the case is placed on the half containing the electrode stack and joined by welding the seam. The battery is completed through addition of an electrolyte solution through the fill port which is then sealed with a metal ball or plate placed over the hole and welded in place to render the enclosure hermetic.

The methods provided for constructing high energy density batteries can be used to assemble batteries directly into an apparatus, in particular, into an implantable medical device. The methods provided tier constructing high energy density batteries can also be used to increase the volumetric energy density of a battery. The methods provided for constructing high energy density batteries can also be used to reduce the size of a battery having a desired energy density.

5.3 Methods for Constructing Anode and Cathode Layers

In one embodiment, the anode layer can comprise a lithium or other group IA metal or alloy thereof.

In another embodiment, the cathode layer can be prepared with discrete active materials that are then used in combination within the electrode stack. These materials can include two or more different cathode active materials such as a mixture of a fluorinated carbon or halogenated carbon material combined with one or more transition metal oxides or sulfides selected from the group consisting of Ag₂O, Ag₂O₂, CuF₂, Ag₂CrO₄, MnO₂, V₂O₅, TiS₂, Cu₂S, FeS, FeS₂, silver vanadium oxide, copper vanadium oxide, copper oxide, and copper silver vanadium oxide.

The mixed materials can be in the form of inter-dispersed powders as described by Weiss (U.S. Pat. No. 5,180,642) pressed into common cathode plates or cathode plates prepared with discrete active material that are then used in combination within the electrode stack as taught by Gan (U.S. Pat. No. 6,607,861). Cathode plates can be formed using methods well known in the art. e.g., by the pressing of the powdered active material and suitable binder or through formation of a rolled or pressed cathode sheet as described by Takeuchi (U.S. Pat. No. 5,435,874).

In yet another embodiment, the high energy density battery can comprise a rechargeable or secondary system. Such electrochemical systems include but are not limited to lithium ion, lithium ion polymer, thin film solid state lithium, nickel cadmium, nickel metal hydride, and lead acid.

5.4 Methods for Connecting Leads, Seals, and Other Elements

Methods for effecting mechanical and electrical connections used in the present invention are common known in the medical battery industry. Metal-to-metal connections required for current conducting tabs, pins, and leads can be made, for example, by any connection method known in the art, e.g., resistance spot welding, laser welding, ultrasonic welding, soldering, brazing or mechanically crimping.

The insulative glass-to-metal seal is well known in the state of the art and available as a sub-component from a variety of commercial sources including Fusite, Teknaseal, and Hermetic Seal Technology Inc. The glass-to-metal seal can be located in the header assembly or in the case wall. Alternatively, a seal utilizing a ceramic or polymer material can be used to enable the insulated electrical connection.

Completing the battery enclosure comprises sealing the case lid or header to the case when a deep-drawn construction is used or joining the two halves if the enclosure utilizes a clam-shell design. Various techniques known in the art that can be employed to make this seal include, but are not limited to laser welding, ultrasonic welding. TIG welding, or through the use of a sealant such as an epoxy.

5.5 Devices Powered by the High Energy Density Battery

The high energy density battery provided by the invention can provide implantable medical devices with extended operation time and/or higher power capability. Longer running time reduces the frequency of battery changes which require invasive medical procedures. Higher power allows the devices to employ additional features beneficial to the user such as self-diagnostic telemetry or higher or more frequent electrical pulses for cardiac defibrillation.

An apparatus comprising a high energy density battery is provided. The apparatus can comprise an electrically powered device and a high energy density battery operatively connected to the electrically powered device.

A method for constructing an apparatus comprising the high energy density battery is also provided. In one embodiment, the method can comprise providing an electrically powered device, providing a high energy density battery, and operatively connecting the high energy density battery to the electrically powered device. Methods for operatively connecting batteries to electrically powered devices are well known in the art.

Examples of such an apparatus include, but are not limited, to an apparatus that requires an internal power source. The battery can be used to power electronics associated with a variety of devices such as medical devices that employ an internal power source, including, but not limited to, any device known in the art for the following cardiac rhythm management e.g., cardiac pacemaking, and cardioverter defibrillation), neurostimulation, dispensing or pumping drug or pharmaceutical compositions (e.g., implantable infusion pumps, insulin pumps), diagnostic sensors (e.g., implantable monitoring or sensing devices implanted to record glucose content, oxygen sensor, telemetry); promotion of regeneration and repair (e.g., bone repair, distractive osteogenesis); tissue repair (electrical pulses for regeneration of neurons, connective tissue, etc.), and human interface applications (e.g., paraplegic assist device disposed on the upper palate of the mouth).

An apparatus comprising an electricity-generating device and a high energy density battery operatively connected to the electricity-generating device. The electricity-generating device can be, for example, a photovoltaic array, a DC power supply, or a charging battery.

A method for constructing such an apparatus is also provided in one embodiment, the method can comprise providing an electricity-generating device, providing a high energy density battery, and operatively connecting the high energy density battery to the electricity-generating device. Methods for operatively connecting batteries to electricity-generating devices are well known in the art.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A high energy density battery comprising:
a case comprising an outer surface, an inner surface and a case opening;
a header assembly inserted in the case opening, the header assembly comprising:
an electrical feed-through, the electrical feed-through comprising a terminal pin,
a glass-to-metal seal, and
a surrounding sidewall extending through the case opening to the outer surface and the inner surface of the case, the case opening being sized to receive the header assembly, with the header assembly surrounding sidewall contacting the case opening;
an electrode stack, the electrode stack comprising:
an anode layer, the anode layer comprising electrically conductive, chemically active material, an upper surface, and a lower surface,
an anode lead, the anode lead comprising electrically conductive material, an anode lead origin and an anode lead end, wherein:
the anode lead origin is connected to the anode layer, and
the anode lead end extends into the case and attaches to the terminal pin:
an insulative separator layer, the insulative separator layer comprising an upper surface and a lower surface;
a cathode layer, the cathode layer comprising electrically conductive, chemically active material, an upper surface and a lower surface;
a cathode lead, the cathode lead comprising electrically conductive material, a cathode lead origin and a cathode lead end, wherein:
the cathode lead origin is connected to the cathode layer, and
the cathode lead end extends into the case and attaches to the positive terminal;
a current collecting lead, the current collecting lead disposed between a cathode tab and the terminal pin, wherein the current collecting lead:
is electrically connected across the cathode layer of the electrode stack, and
is insulated with an insulative material; and
an electrolyte solution, the electrolyte solution disposed within the case and contacting the electrode stack.

2. The high energy density battery of claim 1 that is a primary battery.

3. The high energy density battery of claim 1 that is a secondary battery.

4. The high energy density battery of claim 1 wherein the battery case comprises a material selected from the group consisting of nickel, stainless steel, aluminum, titanium, glass and ceramic.

5. The high energy density battery of claim 1 wherein the battery ease is a deep-drawn battery case.

6. The high energy density battery of claim 1 wherein the battery case is a multi-part or clam-shell case.

7. The high energy density battery of claim 1 wherein the battery case is a liner or insulating bag.

8. The high energy density battery of claim 1 comprising a plurality of electrode stacks, wherein the anode layer of one or more electrode stacks of the plurality is connected in series or in parallel to at least one other anode layer of an electrode stack of the plurality, and the cathode layer of one or more electrode stacks of the plurality is connected in series or in parallel to at least one other cathode layer of an electrode stack of the plurality.

9. The high density energy battery of claim 1 wherein the electrode stacks are electrically connected to the current collecting lead.

10. The high density energy battery of claim 1 wherein the header assembly comprises a plurality of electrical feed-throughs.

11. The high density energy battery of claim 1 wherein the lower surface of the insulative separator layer is disposed on the upper surface of the anode layer.

12. The high density energy battery of claim 1 wherein the lower surface of the cathode layer is disposed on the upper surface of the insulative separator layer.

13. The high density energy battery of claim 1 comprising a plurality of electrode stacks.

14. The high density energy battery of claim 1 wherein the anode layer of one or more electrode stacks of the plurality is connected in series or in parallel to at least one other anode layer of an electrode stack of the plurality, and the cathode layer of one or more electrode stacks of the plurality is connected to at least one other cathode layer of an electrode stack of the plurality.

15. The high energy density battery of claim 1 wherein the anode layer comprises a material selected from the group consisting of a group IA metal or an alloy thereof (e.g., lithium, lithium compound), a group IIIA metal or an alloy thereof, and a carbonaceous material carbon, graphite).

16. The high energy density battery of claim 1 wherein the cathode layer comprises an active material selected from the group consisting of a fluorinated carbon material, a halogenated carbon material, a transition metal oxide, a transition metal sulfide, and a lithium insertion compound.

17. The high energy density battery of claim 16 wherein the active material is an inter-dispersed pressed powder.

18. The high energy density battery of claim 16 wherein the transition metal oxide is selected from the group consisting of Ag₂O, Ag₂O₂, CuF₂, Ag₂CrO₄, MnO₂, V₂O₅, silver vanadium oxide, copper vanadium oxide, copper oxide, and copper silver vanadium oxide.

19. The high energy density battery of claim 16 wherein the transition metal sulfide is selected from the group consisting of TiS₂, Cu₂S, FeS, and FeS₂.

20. The high energy density battery of claim 1 wherein the electrode stack is positioned in the case to minimize unused volume within the case.

21. The high energy density battery of claim 1 wherein the electrode stack has a flat, jelly-roll or serpentine configuration.

22. The high energy density battery of claim 1, wherein the battery delivers at least about 20 joules in about 20 seconds or less.

23. The high energy density battery of claim 1, wherein the battery delivers at least about 20 joules at least twice in a period of about 30 seconds.

24. A method for manufacturing a high energy density battery comprising:
a. providing a case;
b. providing an electrode stack assembly, wherein the electrode stack assembly comprises an anode layer, a cathode layer and a layer of separator material;
c. connecting the anode layer to the case with an anode connecting lead;
d. connecting the cathode layer to an insulated terminal pin with a cathode connecting lead;
e. positioning the anode connecting lead and the cathode connecting lead proximate to the center line of the stack and on the radiused or curved side of the stack;
f. electrically connecting the cathode layer to the positive current collecting lead;
g. electrically connecting the anode layer to a current collecting lead, wherein the current collecting lead is of sufficient length to extend from the side of the electrode stack where the connections are made to the opposing side of the stack assembly;
h. electrically connecting the cathode layer to a current collecting lead, wherein the current collecting lead is of sufficient length to extend from the side of the electrode stack where the connections are made to the opposing side of the stack assembly;
i. electrically connecting the positive current collecting lead to the feed-through pin;
j. electrically insulating the feed-through pin with a glass-to-metal seal, thereby providing the feed-through pin with positive polarity;
k. electrically connecting the anode layer to the negative current collecting lead;
l. electrically connecting the current collecting lead to the case, thereby providing the case with negative polarity;
m. electrically connecting the positive current collecting lead to the glass-to-metal seal of the header assembly and to the electrode stack; and
n. attaching the feed-through pin to the case.

25. The method of claim 24 wherein the case comprises an electrolyte fill port, the method additionally comprising:
introducing an electrolyte solution into an electrolyte till port of the case; and
hermetically sealing the electrolyte till port.

26. Use of the method of claim 24 to increase the volumetric energy density of a battery.

27. Use of the method of claim 24 to reduce the size of a battery having a desired energy density.

28. A method for manufacturing a high energy density battery comprising:
providing a case, the case comprising an open portion;
attaching a header assembly to the case, the header assembly comprising:
an electrical feed-through, the electrical feed-through comprising a terminal pin, and
a glass-to-metal seal;
inserting an electrode stack into the case through the open portion, the electrode stack comprising:
an anode layer, the anode layer comprising electrically conductive, chemically active material, an upper surface, and a lower surface,
an anode lead, the anode lead comprising electrically conductive material, an anode lead origin and an anode lead end, wherein:
the anode lead origin is connected to the anode layer, and
the anode lead end extends into the case and attaches to the terminal pin,
an insulative separator layer, the insulative separator layer comprising an upper surface and a lower surface,
a cathode layer, the cathode layer comprising electrically conductive, chemically active material, an upper surface and a lower surface, and
a cathode lead, the cathode lead comprising electrically conductive material, a cathode lead origin and a cathode lead end, wherein:
the cathode lead origin is connected to the cathode layer,
the cathode lead end extends from the case to the terminal pin,
the cathode lead attaches to the terminal pin,
connecting a current collecting lead to the cathode lead and the terminal pin, wherein the current collecting lead is insulated with an insulative material, and wherein the current collecting lead extends across layers of the electrode stack;
placing an electrolyte solution inside the ease;
placing a cover over the open portion of the ease; and
hermetically sealing the cover to the case.

29. The method of claim 28 wherein the cathode layer is rolled or pressed.

30. The method of claim 28 wherein the cathode layer comprises a material formed by pressing or compressing a powdered active material.

31. Use of the method of claim 28 to increase the volumetric energy density of a battery.

32. Use of the method of claim 28 to reduce the size of a battery having a desired energy density.

33. An apparatus comprising:
a. an electrically powered implantable medical device; and
b. the high energy density battery of claim 1 operatively connected to the electrically powered implantable medical device.

34. The apparatus of claim 33 wherein the electrically powered implantable medical device is selected from the group consisting of cardiac rhythm management device, neurostimulation device, pump for dispensing drug or pharmaceutical composition, diagnostic sensor, regeneration and repair device, tissue repair device, and human interface device.

35. A method for constructing an apparatus comprising:
providing an electrically powered device;
providing, the high energy density battery of claim 1; and
operatively connecting the high energy density battery to the electrically powered device.

36. An apparatus comprising:
an electricity-generating device; and
the high energy density battery of claim 1 operatively connected to the electricity-generating device.

37. The apparatus of claim 36 wherein the electricity-generating device is selected from the group consisting of a photovoltaic array, a DC power supply, and a charging battery.

38. A method for constructing an apparatus comprising:
providing an electricity-generating device;
providing the high energy density battery of claim 1; and operatively connecting the high energy density battery to the electricity-generating device.
